(19) United States
(12) Patent Application Publication
(10) Pub. No.: US 2026/0179740 A1
(43) Pub. Date: Jun. 25, 2026

(54) Blockchain-Based System and Method for Patient Data Transfer

(71) Applicants: Cheryl Ann WILSON, Huntsville (CA); Stevie Jewel Keeter, Huntsville (CA)

(72) Inventors: Cheryl Ann WILSON, Huntsville (CA); Stevie Jewel Keeter, Huntsville (CA)

(21) Appl. No.: 19/417,649

(22) Filed: Dec. 12, 2025

Related U.S. Application Data
(60) Provisional application No. 63/736,132, filed on Dec. 19, 2024.

(51) Int. Cl. G16H 10/60; G06F 21/60; G06F 21/62; G06Q 40/08

(52) U.S. Cl. CPC G16H 10/60; G06F 21/602; G06F 21/6245; G06Q 40/08

(57) ABSTRACT

A method including steps of: i) creating a patient file for a patient in a blockchain-secured database of patient information; ii) adding medical information about the patient to the patient file in the blockchain-secured database; and iii) providing a medical product or service to the patient based on the medical information contained in the patient file in the blockchain-secured database.

### RELATED APPLICATION

This application claims priority to the 19 Dec. 2024 filing date of U.S. Provisional Patent Application Ser. No. 63/736,132, which is incorporated herein by reference.

### FIELD OF THE INVENTION

This invention relates to a system and method for managing and communicating patient data.

### BACKGROUND

Existing processes for communicating and processing patient data have a number of inefficiencies and challenges. For example, the current process for Canadian military veterans obtaining a prescription includes complex and fragmented steps, which can lead to limited access or delays.

Veterans must coordinate with multiple entities, including healthcare providers, health insurance companies, and pharmacies or licensed providers. The processes are often paper-based with manual workflows, and involve medical documents and approvals that must comply with strict privacy laws. Due to the complexity of these processes, veterans in remote areas or those with limited digital, legal, and/or medical literacy may face barriers to access.

### SUMMARY OF THE INVENTION

To at least partially overcome some of the limitations and disadvantages of previous systems and methods, in one aspect the present invention provides an end-to-end digital platform that simplifies, secures, and automates the process of obtaining medical treatment, such as a prescription, while ensuring compliance with healthcare and controlled substance regulations.

Key features of the system include:
Centralized Digital Platform: A secure, blockchain-enabled platform utilizing smart contract technology where patients/veterans, healthcare practitioners, pharmacies, licensed providers, and insurance providers such as Medavie Blue Cross can exchange information seamlessly. Patient information is stored in a database where it is available via smart contract initiation to enter prescription information, submit insurance coverage applications, track approvals, and fulfill prescriptions through a single interface.
Automated Document Verification: Use AI or machine learning to verify medical documents and eligibility in real time. Reduce human errors and speed up approvals.
Integrated Compliance Tools: Automatically check whether prescriptions comply with federal and provincial regulations. Generate audit trails for insurance providers to ensure transparency.
Smart Contracts for Reimbursements: Implement smart contracts that trigger direct billing between pharmacies or licensed providers and insurance providers once a veteran/patient order is fulfilled.
Personalized Recommendations: Use AI to suggest appropriate products based on the patient/veteran's medical condition and prescription, ensuring compliance with dosage limits.
Data security: Blockchain technology secures sensitive medical and transaction data, ensuring it's immutable and accessible only by authorized parties.
Accessibility Features: User-friendly interfaces for veterans with varying levels of digital literacy. Support for remote or underserved areas through telemedicine integration.

The invention may be used to simplify and increase the security of a wide range of multiparty medical transactions, including transactions between medical practitioners, patients, insurers, and pharmacies related to the filling of prescriptions.

Further aspects of the invention include:

A method, which optionally incorporates one or more features of any one or more of the preceding and/or following aspects, comprising: i) creating a patient file for a patient in a blockchain-secured database of patient information; ii) adding medical information about the patient to the patient file in the blockchain-secured database; and iii) providing a medical product or service to the patient based on the medical information contained in the patient file in the blockchain-secured database.

A method, which optionally incorporates one or more features of any one or more of the preceding and/or following aspects, further comprising: reviewing the patient file in the blockchain-secured database to confirm insurance coverage for the patient for the medical product or service.

A method, which optionally incorporates one or more features of any one or more of the preceding and/or following aspects, further comprising: updating the patient file in the blockchain-secured database to confirm the insurance coverage for the patient for the medical product or service.

A method, which optionally incorporates one or more features of any one or more of the preceding and/or following aspects, further comprising: updating the patient file in the blockchain-secured database to confirm that the medical product or service has been provided to the patient.

A method, which optionally incorporates one or more features of any one or more of the preceding and/or following aspects, wherein the medical information comprises a diagnosis.

A method, which optionally incorporates one or more features of any one or more of the preceding and/or following aspects, wherein the medical information comprises a prescription for the medical product or service.

A method, which optionally incorporates one or more features of any one or more of the preceding and/or following aspects, wherein the medical product or service comprises a drug.

A method, which optionally incorporates one or more features of any one or more of the preceding and/or following aspects, wherein the medical product or service comprises a controlled substance.

A method, which optionally incorporates one or more features of any one or more of the preceding and/or following aspects, wherein the patient file is created by a first entity; wherein the medical information is added to the patient file by a second entity different from the first entity; and wherein the medical product or service is provided to the patient by a third entity different from the first entity and different from the second entity.

A method, which optionally incorporates one or more features of any one or more of the preceding and/or following aspects, wherein the first entity is a health authority.

A method, which optionally incorporates one or more features of any one or more of the preceding and/or following aspects, wherein the second entity is a medical clinician.

A method, which optionally incorporates one or more features of any one or more of the preceding and/or following aspects, wherein the third entity is a pharmacy.

A method, which optionally incorporates one or more features of any one or more of the preceding and/or following aspects, wherein the patient file is reviewed and updated to confirm the insurance coverage by a fourth entity that is different from the first entity, different from the second entity, and different from the third entity.

A method, which optionally incorporates one or more features of any one or more of the preceding and/or following aspects, wherein the fourth entity is an insurer.

A method, which optionally incorporates one or more features of any one or more of the preceding and/or following aspects, wherein creating the patient file in the blockchain-secured database initiates a first smart contract or first stage of a smart contract; wherein adding the medical information to the patient file in the blockchain-secured database completes the first smart contract or first stage of the smart contract, and initiates a second smart contract or second stage of the smart contract; wherein updating the patient file in the blockchain-secured database to confirm the insurance coverage completes the second smart contract or second stage of the smart contract, and initiates a third smart contract or third stage of the smart contract; and wherein updating the patient file in the blockchain-secured database to confirm that the medical product or service has been provided to the patient completes the third smart contract or third stage of the smart contract.

A method, which optionally incorporates one or more features of any one or more of the preceding and/or following aspects, wherein completion of the third smart contract or third stage of the smart contract triggers an automatic payment for the medical product or service from the fourth entity.

A method, which optionally incorporates one or more features of any one or more of the preceding and/or following aspects, further comprising: notifying the fourth entity when the second smart contract or second stage of the smart contract is initiated.

A method, which optionally incorporates one or more features of any one or more of the preceding and/or following aspects, further comprising: performing a non-invasive medical test on the patient, wherein the medical information is based at least in part on the non-invasive medical test.

A method, which optionally incorporates one or more features of any one or more of the preceding and/or following aspects, further comprising: performing a diagnostic test on a biological sample from the patient, wherein the medical information is based at least in part on the diagnostic test.

A method, which optionally incorporates one or more features of any one or more of the preceding and/or following aspects, wherein providing the medical product or service to the patient comprises a non-therapeutic transfer of possession of the medical product to the patient.

In a further aspect the present invention resides in a method for verifying medical treatment eligibility for veterans by integrating blockchain for secure storage of medical documents and AI for real-time verification.

In another aspect, the present invention resides in a system that automatically audits prescriptions and dosage limits against regional controlled substance regulations before submission for reimbursement.

In a further aspect, the present invention resides in a process that uses smart contracts to automatically handle payment and reimbursement between veterans, licensed providers, and an insurance provider.

In a still further aspect, the present invention resides in a method that analyzes patient profiles and medical conditions to recommend appropriate products.

In a still further aspect, the present invention resides in a secure, decentralized system for managing medical prescriptions and insurance coverage for patients using blockchain, AI, and smart contracts to streamline approvals, ensure compliance, and enhance data security.

Preferred embodiments of the invention may include one or more of the following: blockchain-secured storage of medical documents; automated AI-powered verification of eligibility and compliance; and smart contract-based authorization, coverage and reimbursement execution.

### BRIEF DESCRIPTION OF THE DRAWINGS

FIG. 1 shows a flow chart of a four phase method in accordance with a preferred embodiment of the present invention.

### DETAILED DESCRIPTION

In a preferred embodiment, the present invention helps to provide simplified healthcare transactions, in which patient data is accessible directly from a database to a medical professional, from the medical professional to an insurance provider, and from the insurance provider to a pharmacy.

The inventors have appreciated that, through the use of blockchain ledgers and smart contracts, transactions between these parties can be made seamless and secure. This preferably helps to reduce fraud, enhances profitability, and better secures sensitive healthcare data.

In one preferred embodiment, the system in accordance with the invention has a blockchain-secured database of patient information that is created through a health authority, such as Health Canada. Upon creating a patient file for a patient, a first smart contract or first stage of a smart contract is made available to a medical professional, such as a doctor or nurse practitioner.

The patient can then visit the medical professional, who can examine the patient in order to diagnose an illness and develop a treatment plan, which may for example include a prescription of a medicine.

The medical professional can then access a secure and user-friendly portal to add diagnosis and prescription information to the patient's file. Once the medical professional enters this information, two things occur: i) the information is entered into the patient's blockchain ledger; and ii) a second smart contract or second stage of the smart contract is initiated for the insurance provider.

The insurance provider is notified of a change to a patient's coverage. The insurance provider accesses the secure and user-friendly portal to confirm the patient information and coverage. This confirmation completes the second smart contract or second stage of the smart contract.

The patient can then reach out to their preferred pharmacy to fill their prescription. The pharmacy then accesses the secure portal (or the portal integrates into a pre-existing management software), and the pharmacy confirms the information and fills the prescription.

Filling the prescription completes the final step of the smart contract, and the patient's ledger is updated.

The system preferably allows for a step-by-step completion of the overall transaction between healthcare parties to secure patient information, prevent excessive and unnecessary handling of patient data, and to prevent intentional fraud or unintentional transcription errors in the patient file.

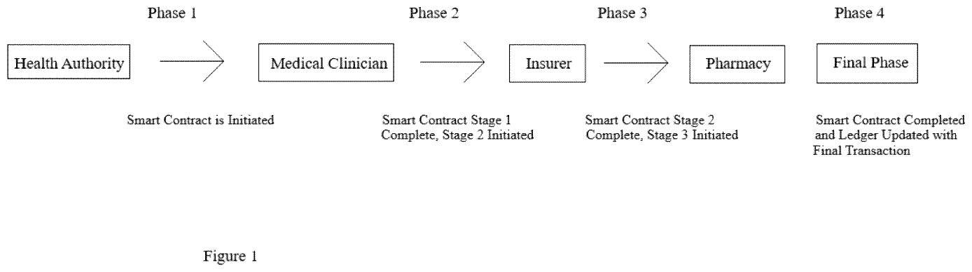

A flow chart showing a preferred implementation of the invention is shown in FIG. 1.

In the first phase, a health authority such as Health Canada or another similar government body or regulated agency initiates the process by creating a patient file in a blockchain secured database. Relying on a government entity such as Health Canada as an anchor for the database is preferred, as this provides a stable, consistent source of information. Creation of the patient file initiates a smart contract, which is made available to a medical clinician such as a doctor or nurse practitioner. The medical clinician then adds diagnosis and prescription information to the patient file. This completes the first phase of the process. At the end of the first phase, the patient ledger is securely stored and the blockchain is updated with diagnosis and treatment information.

In the second phase, the insurer is notified of the patient data update. The insurer then reviews and accepts the blockchain ledger adjustment. This completes the second phase of the process. At the end of the second phase, insurance coverage has been accepted based on the blockchain ledger update.

In the third phase of the process, a pharmacy is contacted by the patient to fill the prescription. The pharmacy reviews the patient file, which now includes the diagnosis and prescription information, as well as the acceptance by the insurer. The pharmacy then provides the prescription to the patient, and updates the blockchain ledger to confirm the prescription has been filled.

In the fourth phase, the smart contract is complete and the ledger is updated. The fourth and final phase demonstrates to all parties a record of each stage of the smart contract having been completed. Specifically, all parties to the contract can securely access the ledger to see the chain of transactions, including: 1) patient record including medical appointment and prescription; 2) insurance recognition of prescription and authorization for coverage; 3) pharmacy acknowledgment of request to fill prescription; and 4) patient pickup of prescription.

Each stage of the process must be completed before the subsequent stages can be completed. This enforces compliance at each stage for accurate information for the next stage to initiate.

The invention has a number of advantages. For example, use of blockchain technology allows for secure data transfer, and provides a list of transactions/data, timestamps, and a cryptographic hash of the previous block.

The invention can also reduce costs, using smart contracts for automation, reduced manpower, and lower overhead.

The invention can also help prevent fraud, with fewer touchpoints, automation, and streamlined movement of information.

Optionally, the invention may further include one or more of the following:
Veteran or Patient Registration: Veterans register on the platform, providing their VAC health ID and personal details. The system uses blockchain to create a secure, tamper-proof profile.
Medical Document Upload: Veterans or patients or healthcare providers upload the medical document to the platform. AI verifies the document's validity, dosage, and compliance with medical laws referring to dosages and controlled substance limits.
Approval Workflow: The system forwards the document to Medavie Blue Cross or similar insurance provider for approval. Smart contracts handle status updates and flag issues, if any.
Product Ordering: Approved veterans access a catalog of compliant products from licensed providers. Blockchain secures transaction records and tracks order fulfillment.
Reimbursement Execution: Pharmacies or Licensed providers are paid directly by Medavie Blue Cross or similar insurance provider via a smart contract once the veteran confirms receipt of the product or the patient has picked up the prescription.
Compliance and Reporting: The platform generates real-time compliance reports for regulators and VAC.

It will be understood that, although various features of the invention have been described with respect to one or another of the embodiments of the invention, the various features and embodiments of the invention may be combined or used in conjunction with other features and embodiments of the invention as described and illustrated herein.

The invention is not limited to the specific preferred embodiments that have been illustrated and described. For example, in some embodiments of the invention the insurer may be omitted from the process, with payment for the medical product or service being made directly by the patient.

Although this disclosure has described and illustrated certain preferred embodiments of the invention, it is to be understood that the invention is not restricted to these particular embodiments. Rather, the invention includes all embodiments which are functional equivalents of the specific embodiments and features that have been described and illustrated herein.

We claim:

1. A method comprising:
i) creating a patient file for a patient in a blockchain-secured database of patient information;
ii) adding medical information about the patient to the patient file in the blockchain-secured database; and
iii) providing a medical product or service to the patient based on the medical information contained in the patient file in the blockchain-secured database.

2. The method according to claim 1, further comprising:
reviewing the patient file in the blockchain-secured database to confirm insurance coverage for the patient for the medical product or service.

3. The method according to claim 2, further comprising:
updating the patient file in the blockchain-secured database to confirm the insurance coverage for the patient for the medical product or service.

4. The method according to claim 3, further comprising:
updating the patient file in the blockchain-secured database to confirm that the medical product or service has been provided to the patient.

5. The method according to claim 1, wherein the medical information comprises a diagnosis.

6. The method according to claim 1, wherein the medical information comprises a prescription for the medical product or service.

7. The method according to claim 1, wherein the medical product or service comprises a drug.

8. The method according to claim 1, wherein the medical product or service comprises a controlled substance.

9. The method according to claim 4, wherein the patient file is created by a first entity;
wherein the medical information is added to the patient file by a second entity different from the first entity; and
wherein the medical product or service is provided to the patient by a third entity different from the first entity and different from the second entity.

10. The method according to claim 9, wherein the first entity is a health authority.

11. The method according to claim 9, wherein the second entity is a medical clinician.

12. The method according to claim 9, wherein the third entity is a pharmacy.

13. The method according to claim 9, wherein the patient file is reviewed and updated to confirm the insurance coverage by a fourth entity that is different from the first entity, different from the second entity, and different from the third entity.

14. The method according to claim 13, wherein the fourth entity is an insurer.

15. The method according to claim 13, wherein creating the patient file in the blockchain-secured database initiates a first smart contract or first stage of a smart contract;
wherein adding the medical information to the patient file in the blockchain-secured database completes the first smart contract or first stage of the smart contract, and initiates a second smart contract or second stage of the smart contract;
wherein updating the patient file in the blockchain-secured database to confirm the insurance coverage completes the second smart contract or second stage of the smart contract, and initiates a third smart contract or third stage of the smart contract; and
wherein updating the patient file in the blockchain-secured database to confirm that the medical product or service has been provided to the patient completes the third smart contract or third stage of the smart contract.

16. The method according to claim 15, wherein completion of the third smart contract or third stage of the smart contract triggers an automatic payment for the medical product or service from the fourth entity.

17. The method according to claim 15, further comprising:
notifying the fourth entity when the second smart contract or second stage of the smart contract is initiated.

18. The method according to claim 1, further comprising:
performing a medical test on the patient, wherein the medical information is based at least in part on the medical test.

19. The method according to claim 1, wherein providing the medical product or service to the patient comprises a physical transfer of the medical product to the patient.

20. The method according to claim 16, wherein the medical information comprises a diagnosis;
wherein the medical information comprises a prescription for the medical product or service;
wherein the medical product or service comprises a drug;
wherein the first entity is a health authority;
wherein the second entity is a medical clinician;
wherein the third entity is a pharmacy;
wherein the fourth entity is an insurer;
wherein providing the medical product or service to the patient comprises a physical transfer of the medical product to the patient;
wherein the method further comprises:
performing a medical test on the patient, wherein the medical information is based at least in part on the medical test; and
notifying the fourth entity when the second smart contract or second stage of the smart contract is initiated.
